(19) United States
(12) Patent Application Publication
(10) Pub. No.: US 2023/0375556 A1
(43) Pub. Date: Nov. 23, 2023

(54) PROTEIN DETECTION AND TRACKING USING NUCLEOSIDE TAGS

(71) Applicant: MGI Tech Co., Ltd., Shenzhen (CN)

(72) Inventors: Snezana Drmanac, Los Altos Hills, CA (US); Radoje Drmanac, Los Altos Hills, CA (US); Matthew J. Callow, San Jose, CA (US)

(21) Appl. No.: 18/247,124

(22) PCT Filed: Oct. 12, 2021
(86) PCT No.: PCT/CN2021/123223

§ 371 Date: Mar. 29, 2023

Related U.S. Application Data
(60) Provisional application No. 63/090,652, filed on Oct. 12, 2020.

(51) Int. Cl. G01N 33/58; G01N 33/68

(52) U.S. Cl. CPC G01N 33/582; G01N 33/68

(57) ABSTRACT

Provided are methods and reagents for detecting polypeptides using nucleosides or nucleoside analogs as tags. In particular, a tagged polypeptide is contacted with a binding reagent (such as an antibody) that binds specifically to the nucleoside tag portion of the tagged polypeptide. The binding of the binding reagent to the nucleoside tag portion of the tagged polypeptide is then detected, thereby quantifying or localizing the tagged polypeptide. Provided is a variety of uses of this technology. For example, the tag specific antibodies can be presented in an array that is suitable for quantifying or characterizing a number of tagged proteins in a number of liquid samples. The technology can also be used to track a large number of tagged proteins in vivo, for example, by multiplex immunohistochemistry.

### BACKGROUND OF THE INVENTION

Biological research and medical diagnostics often use molecular tags that are attached to a protein of interest. These tags allow detection of the protein or its binding partners. Different tagging strategies have been developed that use either direct tagging of proteins with a detectable label or use of labelled antibodies that specifically bind the proteins. However, these approaches have drawbacks. Direct labelling of proteins with fluorophore dyes or by using fusion tags for attaching an organic fluorophore to a protein of interest is widely used, but the large size of these tags can influence protein function and localization. Additionally, use of fluorescent molecules, such as fluorescein, can lead to background fluorescence. Other tagging methods have been shown to induce toxicity or fail to provide sufficient sensitivity and specificity.

### SUMMARY OF THE INVENTION

This disclosure provides methods and reagents for detecting polypeptides using nucleosides or nucleoside analogs as tags. In particular, a tagged polypeptide is contacted with a binding reagent (such as an antibody) that binds specifically to the nucleoside tag portion of the tagged polypeptide, thereby quantifying or localizing the tagged polypeptide. This technology has advantages over other ways of tagging protein.

In general terms, this disclosure provides a method of determining location of a protein in a biological sample. This is done by attaching a tag to the protein to form a tagged protein, wherein the tag is a nucleoside or nucleoside analog; contacting the biological sample with the tagged protein; then identifying and determining location of the protein in the sample by contacting the sample with a binding reagent that binds specifically to the tag.

The tag is a nucleoside, a nucleoside analog, a nucleotide, a naturally occurring nucleoside modified at the 3′ —OH, or other analogs described below. The binding reagent may contain or be attached to a fluorescent group, which makes the binding reagent fluorescent. In this case, the location of the tagged protein in the sample is determined by obtaining an image of fluorescence emitted from the florescent group on the binding reagent.

Various applications of the technology put forth this disclosure include the following: a method of flow cytometry or cell sorting, wherein the biological sample is a sample of cells; a method of immunohistochemistry, wherein the biological sample is a tissue sample; and a method of enzyme linked immuno-assays (ELISA), or producing a western blot. The method may be multiplex, and may be used for tracking one or more proteins in vivo.

Particular methods for tracking proteins in vivo according to this disclosure may include the following steps: obtaining a plurality of protein; administering the tagged proteins to a subject either as a mixture, simultaneously, or sequentially; obtaining a biological sample from the subject; and determining the presence, quantity, and/or location of each of the tagged proteins in the biological sample by contacting the biological sample with binding reagents specific for each of the tags on the tagged proteins. The subject may be any animal or vertebrate, such as an experimental mouse, or a human subject for purposes of diagnosis and/or treatment. The biological sample may be a tissue section prepared for immunohistochemistry (for example, as a paraffin imbedded tissue slice). In this case, each of the specific binding reagents specific for a nucleoside or analog used to determine location of the respective tagged proteins in the tissue section. Optionally, this can be implemented as a multiplex method, in which at least some of the tagged proteins have been tagged with two or more different nucleoside or nucleoside analogs, wherein a particular combination of the nucleoside or nucleoside analogs uniquely identifies each of the tagged proteins.

Another implementation of the technology for tracing proteins in vivo is an iterative or sequential multiplex method. This include multiple iterations or cycles of immunohistochemistry: for example, contacting the tissue section with a set of tag-specific binding reagents that collectively bind some but not all of the different nucleoside tags attached to proteins in the tissue section; locating tagged proteins in the tissue section that bear tags for which the binding reagents in the set are specific; and removing the binding reagents from the tissue sample. One or more additional iterations or cycles may be performed of the contacting, locating, and removing using a different set of binding reagents that contains at least one binding reagent that is specific for a tag that is different from the tags to which the binding reagents were specific in preceding iteration(s) or cycle(s).

The number of iterations or cycles may continue until all of the tagged proteins administered to the subject that may be present in the tissue sample are identified and located. The binding reagents may be removed before the next iteration using a solution that contains a non-physiological salt concentration and/or pH, and/or one or more nucleosides or nucleoside analogs that are not attached to proteins. The binding reagents used to contact the tissue section in each set may be fluorescent (containing or attached to a fluorescent group). In this case, the determining in each set includes obtaining an image of emitted fluorescence from binding reagents bound to the tissue section. The number of proteins that can be detected and localized can be as high as the number of tabs and binding reagents used in each iteration, times the number of iterations performed.

By way of illustration, four different proteins are located during each iteration using four fluorescent binding reagents that are specific for each of the tags attached to the four different proteins, wherein fluorescent emission from each of the binding reagents is optically distinguishable. The images from multiple iterations can then be computationally merged or otherwise processed to compare locations of some or all of the tagged proteins in the tissue sample.

Instead of a tissue section, the biological sample may be a liquid sample, such as plasma or lymph, or solid tissue that has been solubilized. The presence and/or quantity of tagged protein in the liquid sample may be determined using an array of binding reagents on a solid surface. Each position on the array comprises a binding reagent specific for a different tag.

For any of the technology put forth in this disclosure, the binding reagent(s) may be antibodies or antibody fragments, affimers, or other molecules that bind a target antigen in a specific fashion, as listed below.

The identity, quantity, and/or location of tagged protein in the sample can be confirmed by contacting the sample with the binding reagent that binds specifically to the tag before and after modifying the tag enzymatically, chemically, by bleaching, or by other means. For example, a tissue sample is contacted with a binding reagent specific for the tag of a tagged protein located in the sample. After binding and detection, the binding reagent is removed. The tag is then modified or removed using a suitable enzyme or other means. After washing, the sample is again contacted with the binding reagent specific for the same tag. The binding of the reagent the first time is confirmed as authentic if the same reagent fails to bind after the tag is modified or removed.

Other embodiments, aspects, and features of the invention are presented in the description that follows, the accompanying drawings, and the appended claims.

### BRIEF DESCRIPTION OF THE DRAWINGS

FIG. 1 illustrates the synthesis of an amino-reactive N-hydroxysuccinimide (NHS) ester of a nucleoside analog comprising a 3′ —O azidomethyl moiety, thereby preparing the analog for conjugating to a selected protein.

FIGS. 2A, 2B, and 2C provide information for antibody molecules generated in the course of developing this technology that specifically bind various nucleosides and nucleoside analogs in accordance with this disclosure.

### DETAILED DESCRIPTION OF THE INVENTION

### Introduction

Disclosed herein are methods and reagents for detecting a polypeptide by tagging the polypeptide with one or a combination of nucleosides and/or nucleoside analogs, thereby creating a tagged polypeptide. For simplicity, as used herein the term “nucleoside” will be used to describe both nucleosides and nucleoside analogs (as defined hereinbelow), unless otherwise clear from context.

The nucleoside tag(s) used for labeling a specific polypeptide are selected so that the identity of the tag or combination of tags identifies the polypeptide. The method further comprises detecting the tagged polypeptide using binding reagents that specifically bind the tags. Thus, in one approach, the method involves contacting a tagged polypeptide, comprising a polypeptide portion and a nucleoside tag portion, with a binding reagent that binds to the nucleoside tag portion of the tagged polypeptide. Detection of the polypeptide occurs through detection of the binding of the binding reagent to the nucleoside tag portion of the tagged polypeptide. In some approaches, the method involves making a tagged polypeptide by coupling at least one nucleoside or nucleoside analogs to a polypeptide, and detecting the polypeptide using a binding reagent or reagents that specifically bind a nucleoside or nucleoside analog.

In some approaches, the nucleoside tag is a naturally occurring nucleoside. In some approaches, the binding reagent recognizes and binds to the nucleobase of the nucleoside. In other approaches, the nucleoside tag is a nucleoside analog. A nucleoside analog may comprise one or more modifications relative to a naturally occurring nucleoside. For example and not for limitation, modifications may be in the nucleobase, the sugar moiety, or involve the addition of a phosphate moiety. In some embodiments the nucleoside analog differs from a naturally occurring (unmodified) molecule by addition of a group to the 3′-OH of ribose or deoxyribose. In some cases, the group is a 3′ —OH blocking group that can be removed (cleaved) from the deoxyribose (e.g., 3′ —O-azidomethyl).

In some approaches the binding reagent (e.g., an antibody or aptamer) used for carrying out this technology** is bound to a detectable label allowing direct detection of the binding of the binding reagent to the nucleoside tag portion of the tagged polypeptide. In some approaches, detection may occur indirectly where a labelled secondary binding reagent (e.g. secondary antibody) binds a primary binding reagent (e.g. primary antibody) that binds the nucleoside tag portion of the tagged polypeptide.

### Advantages of Using Nucleosides as Protein Tags

The methods of identifying and tracking proteins using a tag comprising a nucleoside or nucleoside analog put forth in this disclosure are superior to other labelling methods and other types of tags for several reasons. Such reasons include the following:
A large number of proteins can be tracked simultaneously in the same experiment by tagging each protein with its own unique nucleoside or nucleoside analog
Using a nucleoside tagged protein followed by a fluorescent labeled binding reagent amplifies the detection signal
In spite of their similarity, the different nucleoside or nucleoside analogs can be distinguished from each other without difficulty using a specific antibody or other binding reagent
Different nucleosides or nucleoside analogs used for tagging different proteins will all have similar physicochemical properties, such as size, charge, and reactivity. This helps ensure that the respective tag won't influence behavior of different proteins in different ways
Denaturation of a tagged protein will not interfere with antibody binding, because the nucleoside tag will still be intact and associated with the protein after denaturation
Determination and tracking of each protein can be confirmed easily by modifying the nucleoside tags chemically or using an enzyme, followed by a second round of staining with the same binding reagent
   Uses of the Nucleoside Tagging Technology of this Disclosure

The method of the present disclosure can be used in a wide variety of applications in the fields of biological research and diagnostic medicine. For example, the method described herein can be used to study protein localization, protein function, and protein interactions. A variety of detection techniques may be used to detect tagged proteins, including immunohistochemistry (IHC), flow cytometry (e.g., fluorescence-activated cell sorting), enzyme linked immuno-assays (ELISA), and western blots.

### Localization of Tagged Protein(s) in Tissue Sections and Other Biological Samples

In some approaches, the presence or localization of a protein of interest in a sample such as a tissue may be determined. For example, in the case of IHC, the sample may be fixed with formalin and paraffin-embedded and cut into sections for viewing by light microscopy.

For example, a protein (e.g., interferon lambda 3, IFNL3) can be tagged with a nucleoside (e.g., deoxyadenosine) and detected using a labeled binding agent that recognizes deoxyadenosine (e.g., an anti-deoxyadenosine nanobody conjugated to rhodamine dye). The tagged protein is injected into a subject (e.g., a mouse). After a period of time the mouse is sacrificed, tissues obtained, fixed and sectioned. The presence, absence or location of IFNL3 in tissue sections is determined using immunohistochemistry and the labeled anti-adenosine nanobody.

In a related example, a biological fluid (e.g., cerebrospinal fluid) may be collected from the mouse described above, and contacted with the labeled anti-deoxyadenosine nanobody under conditions in which the nanobody binds tagged IFNL3, if present in the fluid.

For example, a protein (e.g., an interferon lambda 3 variant that is modified so that it binds the IFNL3 receptor but is not internalized) can be tagged with a nucleoside (e.g., deoxycytosine) and detected using a labeled binding agent that recognizes deoxycytosine (e.g., an anti-deoxycytosine nanobody conjugated to fluorescein dye). The tagged protein is injected into a subject (e.g., a mouse). After a period of time, a biological fluid (e.g., lymph) is collected from the mouse and flow cytometry is used to count cells the number of cells in lymph that display IFNL3 on the cell surface.

In a related approach, discussed in greater detail below, two different tagged proteins can be detected in the same sample. For illustration, deoxyadenosine-tagged IFNL3 and deoxycytosine-tagged interferon lambda 3 variant (both as described above) can be injected into a mouse. After a period of time, a tissue section can be obtained and stained with anti-deoxycytosine nanobody conjugated to fluorescein and anti-deoxyadenosine nanobody conjugated to rhodamine, and the presence, location or absence of the two proteins in the tissue can be determined by IHC.

### Multiplexing to Detect and Track Several Tagged Proteins in a Sample

As illustrated above, in some applications multiplexing is used to follow multiple proteins. For example, four proteins (e.g., IFN-alpha, IFN-beta, IFN-gamma, and IFNL3) can each be tagged with a unique nucleotide selected from A, T, G and C.

TABLE 1

| Labeled binding | | Protein to which tag | |
|---|---|---|---|
| reagent | Tag | is coupled | Results |
| (rhodamine labeled- | A | IFN-alpha | Detecting rhodamine signal identifies presence |
| anti-IFN-alpha | | | or location of IFN-alpha |
| fluorescein labeled | T | IFN-beta | Detecting fluorescein signal identifies presence |
| anti-IFN-beta | | | or location of IFN-beta |
| cyanine labeled anti- | G | IFN-gamma | Detecting cyanine signal identifies presence or |
| IFN-gamma | | | location of IFN-gamma |
| pyrene labeled anti- | C | IFNL3 | Detecting pyrene signal identifies presence or |
| IFNL3 | | | location of IFN-alpha |

The proteins can be administered to the mouse and detected by IHC using four uniquely labeled antibodies (rhodamine labeled anti-IFN-alpha, fluorescein labeled anti-IFN-beta, cyanine labeled anti-IFN-gamma, and pyrene labeled anti-IFNL3). In this example four proteins can be tracked using four antibodies. Although this example makes reference to naturally occurring nucleosides, modified nucleosides and nucleotides (e.g., 3′-O-azidomethyl-2′-deoxyguanine, a 3′-O-azidomethyl-2′-deoxyadenine, a 3′-O-azidomethyl-2′-deoxycytosine, or a 3′-O-azidomethyl-2′-deoxythymine) can also be used as discussed in detail below.

### Multiplexing to Detect Multiple Polypeptides Simultaneously

As illustrated above, by selecting different nucleosides or nucleoside analogs as tags, the method can be used for simultaneous or consecutive detection of multiple different polypeptides in a sample (“multiplexing”). In one approach, each polypeptide is coupled with a different nucleoside or nucleoside analog and the binding reagents that specifically recognize and bind the corresponding nucleoside tag portion can be differently labelled. See TABLES 2 and 3. For example, in the case of fluorescent labels, analyses can then be conducted in which different detectable labels are excited at (and/or emit at) different wavelengths.

TABLE 2

| | Tag and specificity of binding reagent | | | |
|---|---|---|---|---|
| | Deoxyribo- | 3′-nitrobenzyl | 3′-azidomethyl | 3′-nitrobenzyl |
| Protein | adenosine | deoxy-riboadenosine | deoxy-riboguanosine | deoxy-ribocytosine |
| Protein 1 | ✓ | | | |
| Protein 2 | | ✓ | | |
| Protein 3 | | | ✓ | |
| Protein 4 | | | | ✓ |

Four proteins can be distinguished if each is tagged with a different naturally occurring nucleoside, but that by using modified nucleosides a much larger number of proteins can be detected,

In a related approach, each polypeptide is coupled with a unique combination of at least two different nucleosides and/or nucleoside analogs and the binding reagents that specifically recognize and bind the corresponding nucleoside tag portion can be differently labelled.

TABLE 3

| | Tag and specificity of binding reagent | | | |
|---|---|---|---|---|
| | Deoxyribo- | Deoxyribo- | Deoxyribo- | Deoxyribo- |
| Protein | adenosine | thymine | guanosine | cytosine |
| Protein 1 | ✓ | | | |
| Protein 2 | | ✓ | | |
| Protein 3 | | | ✓ | |
| Protein 4 | | | | ✓ |
| Protein 5 | ✓ | | ✓ | |
| Protein 6 | | ✓ | | ✓ |
| Protein 7 | ✓ | | ✓ | |
| Protein 8 | | | ✓ | ✓ |

By using combinatorial tagging as illustrated in TABLE 3, eight (8) proteins may be detected using only four binding reagents (e.g., antibodies). Although this example makes reference to naturally occurring nucleosides, modified nucleosides and nucleotides can also be used.

Although the present multiplexing method allows a large number of different proteins to be detected in the same sample, it could still be challenging to assign unique labels to each binding reagent such that a large number of binding reagents could be simultaneously detected and distinguished. For example, in the case of fluorescent dyes, dye combinations with overlapping emission spectra may be difficult to distinguish. This issue also arises when multiple labels are assigned to the same protein (as illustrated in Table 3, and it may be difficult to analyze two different signals that are superimposed on each other. In some approaches, this is addressed by staining with a subset of binding reagents, detecting signal from those binding reagents, removing the binding reagents and carrying out additional rounds of staining and removal. These issues are discussed below in the section captioned “Preparation and use of removable antibodies.”

### Quantitation or Characterization of Tagged Proteins Using Specific Binding Reagents in an Array

The technology provided in this disclosure can be used to follow a large number of proteins simultaneously in various types of reactions in vitro or in vivo.

By way of illustration, suppose a user wishes to study the interaction of a plurality (say ten) different cytokines with cells of the immune system. Each of the ten cytokines is tagged with its own nucleoside or nucleoside analog, and then combined into a single mixture. For purposes of in vitro study, the labelled cytokine is combined with a culture of immune cells, under conditions where they are expected to react with the cells. Supernatant of the culture is then recovered for quantitation. For purposes of in vivo study, the cytokine mixture is administered to an experimental animal or other subject. A liquid biological sample (such as plasma, lymph, or a solid tissue that has been solubilized) is then recovered from the subject at a later time for quantitation. Alternatively, rather than combining the cytokines into a single mixture to start, individual tagged cytokines or subgroups of cytokines may be contacted with the immune cells in vitro or administered in vivo in a sequential fashion.

To perform the quantitation, antibodies or other antigen binding reagents specific for each of the nucleosides are arrayed separately on a suitable surface to detect and/or quantify each of the tagged proteins in the sample. For example, ten rows of a microtiter plate can be coated with ten different antibodies specific respectively for the nucleoside or nucleoside analogs on each of the ten cytokines. This would form a universal array for detecting tagged proteins in recovered samples. Multiple columns in this setup can be used to quantitate successive biological samples recovered over various periods of time, or for a plurality of replicates or reactions run under different conditions.

Each of the recovered samples is then placed in each of the rows in the same column of the microtiter plate. The amount of tagged cytokine in each of the wells can be determined quantitatively, for example, in a subsequent step using a soluble form of the corresponding nucleoside-specific antibody with a fluorescent label. In this setup, the fluorescent label could be the same for all of the antibodies, since each tagged cytokines is being quantitated in a different row. Intensity of the fluorescence in each of the rows will be proportional to the amount of the corresponding tagged cytokine in the corresponding sample. The amount of tagged cytokine that was consumed in the reaction or bound to a cell or tissue in vitro or in vivo can be determined by subtraction from the intensity observed.

A similar approach can be used to determine whether any of the tagged protein has bound to other proteins in the course of its interaction with the immune cells. For example, the tagged protein is captured from a biological sample using an array of antibodies specific for the tag. Rather than developing the array with a second layer of antibodies specific for the tag, labeled antibodies can be used that are specific for the ligand or binding partner of the tagged protein. Wells that stain positively with the antibody indicate that the protein bound to a position on the array via its tag has bound the protein recognized by the developing antibody

A tagged protein that has been through a reaction can also be recovered for further analysis. For example, aliquots of a biological sample obtained from an in vitro or in vivo experiment are contacted with affinity beads expressing antibody specific for each tagged protein. The protein is then eluted from affinity beads under mild conditions (for example, using free small molecule nucleoside or analog in a salt solution) for further analysis.

General information on the preparation and use of antibody arrays is described by Chaga et al., 2008. “Antibody arrays for determination of relative protein abundances,” Methods in Molecular Biology. 441:129-51. doi:10.1007, 978-1-60327-047-2_9. ISBN 978-1-58829-679-5.

### Sequential Multiplex Immunocytochemistry to Follow a Large Number of Tagged Proteins In Vivo

The technology provided in this disclosure can be used to detect or locate a plurality of proteins that are administered to an experimental animal or other subject in multiple cycles of immunohistochemistry and antibody removal of the same sample. A large number of proteins can be detected

Using traditional methods, the number of proteins that can be tracked in vivo is limited by potential overlap of the detection method. For example, take the case where proteins labelled with different fluorescent dyes are injected into an experimental animal. A tissue section is recovered at a later time and prepared for IHC. The position of each of the proteins in the section is determined by imaging each of the fluorescent labels. The tracking is limited to about four differently labelled proteins in the same animal, because of fluorescence overlap.

Using the technology put forth in this disclosure, there is no such limitation. The only limitation is the number of distinguishable tags or tag combinations that are used to tag the proteins of interest. The user simply determines a subset of the proteins using removable tag-specific binding reagents at a time. The user then removes the binding reagents from the sample being assessed, and determines additional subsets of tagged proteins in the same sample in subsequent iterations of binding, identification, and removal.

For example, starting with a tissue section containing 20 tagged proteins, the technology may be implemented as follows: The sample is contacted with a first set of four different antibodies specific for four of the nucleoside tags on the tagged proteins, each with a different fluorescent label. The first four labels are imaged and quantified. The antibodies are then removed from the tissue section under mild conditions, thereby preserving the tissue. The tissue section is then contacted with a second set of four labelled antibodies that are specific for four different tags not recognized by the first set of antibodies. In this way, the user can locate and assess four of the tagged proteins at a time in each of five iterations, until all of the 20 tagged proteins are detected and localized. There will be five images of the tissue sample, corresponding to each of the iterations. These images can be computationally combined or otherwise processed so that all 20 of the tagged proteins are determined.

As a result, location and quantitation of all of the tagged proteins is accomplished in the same tissue section harvested from the same animal.

### Confirming a Binding Reaction by Chemically Modifying the Nucleoside Tag

Any of the protein detection methodology provided in this disclosure can be coupled with an additional procedure that verifies the specificity of the binding reaction, thereby confirming the presence, quantity, and/or location of the tagged polypeptide In the sample.

After the tagged protein has been detected or quantified in a sample with a tag-specific binding reagent according to this disclosure, the verification proceeds as follows: The sample is subject to any of a variety of known chemical or enzymatic reactions to effect a transformation of the nucleoside or nucleoside analog on the detected protein, thereby producing a derivative thereof that is not recognized by the binding reagent.

In one example, the 3′ reversible terminator group is o-azidomethyl, which can be unblocked with Tris(hydroxypropyl) phosphine (THPP) for 2 minutes at 55° C. In another example, the nitrobenzyl group can be unblocked with UV light to convert the 3′ blocking group back to an hydroxyl group. (Metzker M L, et al. Termination of DNA synthesis by novel 3′-modified deoxyribonucleoside triphosphates. Nucleic Acids Res. 1994; 22:4259-4267). The 3′ phosphate groups can be removed by enzymatic cleavage mechanisms such as with phosphatases (e.g., shrimp alkaline phosphatase, calf-intestinal phosphatase, antarctic phosphatase and T4 polynucleotide kinase). In each of these examples the removal of the modification is expected to prevent significant binding of the specific antibody raised against that modified nucleoside before the modification.

The modification reaction is done under conditions that preserve the general characteristics of the sample. The sample is then subject to the same detection or quantitation reaction using the same tag-specific antibody. Following the transformation, the tag-specific antibody should no longer bind to the previously tagged protein, thereby eliminating the signal.

Thus, tagged protein that is detected or quantified with the tag-specific antibody before the transformation, and then no longer detected after the transformation, is confirmed as being genuine. A signal that seems to indicate the presence of tagged protein in the first staining reaction but is still detected after the transformation may be a false positive.

### Methods and Materials for Tagging Proteins

Some aspects of the disclosure relate to a method for detecting a polypeptide(s) in a sample. The method comprises contacting a tagged polypeptide with a binding reagent and detecting the binding of the binding reagent to the nucleoside tag portion of the tagged polypeptide. The tagged polypeptide comprises a polypeptide portion and a nucleoside tag portion, and the binding reagent recognizes and binds to the nucleoside tag portion of the tagged polypeptide. In some approaches, the binding reagent does not bind to the polypeptide portion of the tagged polypeptide. In some approaches, the binding reagent does not bind to the polypeptide portion of the tagged polypeptide in the absence of the nucleoside tag portion.

A tagged polypeptide will comprise a polypeptide portion and a nucleoside tag portion. In certain embodiments, the tagged polypeptide comprises multiple nucleoside tags, which may be the same or different.

The tagged polypeptide may comprise any polypeptide for which the presence, location and/or protein interaction profile is to be determined. In this context, the terms “polypeptide” and “protein” are used interchangeably. In some embodiments, the polypeptide is a mammalian polypeptide, e.g., a human polypeptide. Exemplary polypeptides include but are not limited to signal transduction proteins (e.g., cell surface receptors, kinases, adapter proteins), nuclear proteins (transcription factors, histones), mitochondrial proteins (cytochromes, transcription factors) and hormone receptors. In some embodiments, the polypeptide is an antibody or an antibody fragment. The polypeptide may be naturally occurring, recombinant, or synthetic, or any combination thereof. In certain embodiments, the polypeptide is a therapeutic polypeptide.

The polypeptide may have any size as long as it allows the user to carry out the technology. For example, the molecular weights of the polypeptide may be in the range of about 500 Da to 15,000 Da. In some embodiments, the polypeptide has a molecular weight of less than about 1500 Da, or less than about 1000 Da. Alternatively, the protein may be 10 kDa, 25 kDa, 50 kDa or more; or between 100 Da and 50 kDa.

### Nucleosides

As used herein, the term “nucleoside” has its normal meaning in the art. A naturally occurring nucleoside comprises a naturally occurring nucleobase (e.g., adenine (A), cytosine (C), guanine (G), thymine (T), or uracil (U)) linked to the 1′ carbon of a five-carbon sugar (e.g., ribose or 2′-deoxyribose). A nucleoside comprising a 2′-deoxyribose has the following formula:

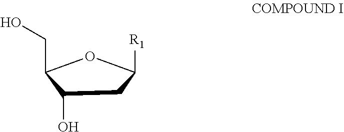

where R₁ is a naturally occurring nucleobase. Naturally occurring nucleosides that may be used for tagging proteins include deoxyadenosine, deoxycytidine, deoxyguanosine, deoxythymidine and their ribose counterparts adenosine, cytidine, guanosine, and thymidine.

### Nucleoside Analogs

In some embodiments, the nucleoside tag portion of the tagged polypeptide comprises a nucleoside analog. Nucleoside analogs differ from naturally occurring nucleosides by a modification of the nucleobase, modification of the five-carbon sugar, and/or the addition of a phosphate moiety. A nucleoside analog comprising a phosphate linked at the 3′ carbon of the sugar moiety is referred to as a “nucleotide.” In some embodiments, a nucleoside analog may comprise two or more modifications relative to a naturally occurring nucleoside. For example, a nucleoside analog may comprise a nucleobase modification and a modification to the five-carbon sugar. Any suitable nucleoside analog comprising a modification may be used. Preferably the modification does not interfere or disrupt the function or activity of the polypeptide to which it is coupled.

Nucleoside analogs are discussed in, e.g., Scheit (1980), Nucleotide Analogs, John Wiley & Son; Uhlman et al. (1990), Chemical Reviews 90:543-584, and Fasman (1989), Practical Handbook of Biochemistry and Molecular Biology, pp. 385-394, CRC Press. Nucleoside modifications have also been described, for example, in Sood et al. (2019), “DNAmod: the DNA modification database”, J. Cheminformat. 23; 11(1):30; Cantara et al. (2001), “The RNA Modification Database, RNAMDB: 2011 update”, Nucleic Acids Res.; 39: D195-D201; Limbach et al. (1994), “Summary: the modified nucleosides of RNA”, Nucleic Acids Res.; 22:2183-2196, Roundtree et al. (2017), “Dynamic RNA modifications in gene expression regulation”, Cell, 169:1187-1200. Other nucleoside analogs suitable for use are described in databases such as the RNAMDB, DNAmod, RMBase, or MODOMICS. Exemplary nucleoside analogs and modifications that are described below.

### Phosphate and Phosphate Analog Moieties

In some embodiments, the nucleoside analog is a nucleotide comprising a phosphate moiety linked to the five-carbon sugar. The phosphate moiety may contain one or more phosphates. In some aspects, the phosphate moiety may comprise 1-12 phosphates (e.g., 1, 2, 3, 4, 5 or more than 5 phosphate groups). For example, the nucleoside analog may be a nucleoside monophosphate, a nucleoside diphosphate or a nucleoside triphosphate.

In some aspects, the nucleoside analog may comprise a phosphate analog moiety. Phosphate analogs include phosphorothioate, phosphorodithioate, alkyl-phosphonate, phosphoroselenoate, phosphorodiselenoate, phosphoroanilothioate, phosphoranilidate, and phosphoramidate, or any other phosphate analog known in the art.

### Nucleobase Modifications

In some embodiments, the nucleoside analog used comprises a nucleobase modification. Modifications to the nucleobase may, for example, include an addition of a methyl group to the 6-atom ring or a substitution of a nitrogen atom with one of carbon. Exemplary nucleobases with modifications include, but are not limited to, hypoxanthine, xanthine, 7-methylguanine, 7-methyladenine, deazapurine (e.g., 7-deazaadenine, 7-deazaguanine), 5-methtylcytosine, 7-methycytosine. Nucleosides comprising nucleobase modifications include, but are not limited to xanthosine, 1-methyladenosine (m1A), N6-methyladenosine (m6A), inosine (I), 5-methylcytidine (m5C), pseudouridine (ψ), dihydrouridine. In some cases the nucleobase portion is not modified. In some cases the nucleoside analog is other than hypoxanthine, xanthine, 7-methylguanine, 7-methyladenine, deazapurine (e.g., 7-deazaadenine, 7-deazaguanine), 5-methtylcytosine, or 7-methycytosine. In some cases, nucleosides comprising nucleobase modifications are other than xanthosine, 1-methyladenosine (m1A), N6-methyladenosine (m6A), inosine (I), 5-methylcytidine (m5C), or pseudouridine (ψ).

A binding reagent may specifically bind to a nucleoside analog by recognizing a nucleoside with a modified nucleobase (e.g., a modified A). A binding reagent that specifically recognizes the modification generally does not bind to a nucleoside with a nucleobase without that modification. For example, binding reagent that binds to an adenosine analog in which nitrogen at position 7 is replaced by methylated carbon may not bind to a naturally occurring and unmodified adenosine that does not comprise a modification to the nucleobase, or may bind less avidly.

### Sugar Modifications

In some embodiments, the nucleoside analog comprises a modification at the five-carbon sugar. In some embodiments, the modification includes the addition of a chemical moiety at the 3′-O position, the 2′-O position, or both the 3′-O position and the 2′-O position of the sugar. In some embodiments the modification (e.g., at the 2′-O position or 3′-O position) contains fewer than 10, e.g., fewer than 5 carbon atoms.

Exemplary chemical moieties include azidomethyl, allyl, aminoalkoxyl, 2-cyanoethyl, alkyl, alkenyl, alkynyl, heteroalkyl, heteroalkenyl, heteroalkynyl, cis-cyanoethenyl, trans-cyanoethenyl, cis-cyanofluoroethenyl, trans-cyanofluoroethenyl, cis-trifluoromethylethenyl, trans-trifluoromethylethenyl, biscyanoethenyl, bisfluoroethenyl, cis-propenyl, trans-propenyl, nitroethenyl, acetoethenyl, methylcarbonoethenyl, amidoethenyl, methylsulfonoethenyl, methylsulfonoethyl, formimidate, form hydroxymate, vinyloethenyl, ethylenoethenyl, cyanoethylenyl, nitroethylenyl, amidoethylenyl, amino, cyanoethenyl, cyanoethyl, alkoxy, acyl, methoxymethyl, aminoxyl, carbonyl, nitrobenzyl, coumarinyl, and nitronaphthalenyl. The alkyl, alkenyl, alkynyl, heteroalkyl, heteroalkenyl, or heteroalkynyl molecules used herein may be substituted or unsubstituted.

In some embodiments, the nucleoside tag portion comprises a nucleoside analog that includes a moiety at the 3′-O position of the sugar. In some embodiments, the moiety is an azidomethyl moiety. In some embodiments, the nucleoside analog comprises a naturally occurring and unmodified nucleobase (e.g., A, G, C, T, or U) and an azidomethyl moiety at the 3′-O position of the sugar. In some embodiments, the nucleoside analog is a nucleotide comprising a naturally occurring and unmodified nucleobase (e.g., A, G, C, T, or U) and an azidomethyl moiety at the 3′-O position of the sugar. In some embodiments, the nucleoside analog is a 3′-O-azidomethyl-2′-deoxyguanine, a 3′-O-azidomethyl-2′-deoxyadenine, a 3′-O-azidomethyl-2′-deoxycytosine, or a 3′-O-azidomethyl-2′-deoxythymine.

Accordingly, in some approaches, the binding reagent specifically recognizes a modification at the sugar, and binds to a 2′-O or 3′-O moiety attached to the sugar. For example, the binding reagent may recognize and bind to an azidomethyl moiety at the 3′-O position of the sugar.

### Nucleoside Analogs Comprising Multiple Modifications

In some embodiments, a nucleoside analog may comprise modifications at one or more positions. For example, a nucleoside analog may have a nucleobase modification and a modification to the sugar, or may have a 2′-O modification and a 3′-O modification. In one embodiment, the sugar modification is a 3′-O-azidomethyl modification.

Additional exemplary nucleoside analogs comprising two or more modifications include 5,2′-0-dimethylcytidine (m5Cm), N4-acetyl-2′-O-methylcytidine (ac4Cm), N6-methyl-N6-threonylcarbamoyladenosine (m6t6A), or N2-2′-O-dimethylguanosine (m2Gm). Synthetic nucleoside analogs comprising one or more modifications may also be used include, for example, didanosine (ddl), vidarabine, BCX4430, cytarabine, gemcitabine, emtricitabine (FTC), lamivudine (3TC), zalcitabine (ddC), abacavir, aciclovir, entecavir, stavudine (d4T), telbivudine, zidovudine (azidothymidine, or AZT), idoxuridine, and trifluridine.

Thus, a binding reagent may recognize and specifically bind a combination of modifications. For example, the binding reagent may recognize a specific 3′-O moiety and/or a nucleobase with a specific modification. In some aspects, nucleoside analogs may be used or selected for the property of being recognized by a specific binding reagent that shows specificity for a particular combination of modifications. Using nucleoside analogs with more than one modification may have the advantage of stronger and more specific binding of the binding reagent and offer more sensitive protein detection.

### Properties of Nucleosides and Nucleoside Analogs

The molecular weights of naturally occurring nucleosides are in the range of about 227 to 283. In certain embodiments, a nucleoside analog used has a molecular weight less than 550, often less than 540, often less than 530, often less than 520, often less than 510, often less than 500, often less than 490, often less than 480, often less than 470, often less than 460, and sometimes less than 450.

Naturally occurring nucleosides typically comprise 9-10 carbon atoms. In certain embodiments, the number of carbon atoms in a nucleoside analog does not exceed that of the naturally occurring nucleoside by more than 1, 2, 3, 4, 5 or 10.

In certain embodiments, where the modification includes the addition of a moiety, for example the addition of a moiety to the sugar, the moiety (including the 3′ oxygen atom or 2′ oxygen atom) has a molecular weight (MW) less than 200, often less than 190, often less than 180, often less than 170, often less than 160, often less than 150, often less than 140, often less than 130, often less than 120, often less than 110, and sometimes less than 100.

The molecular weights of naturally occurring nucleobases are: 135 for adenine; 151 guanine, 126 thymine, and 111 cytosine. In certain embodiments, where the modification includes a nucleobase modification, the modified nucleobase has a molecular weight that does not exceed that of the naturally occurring nucleobase by more than 10, 20, 30, 40, 50, 60, 70, 80, 90, or 100 Da.

### Coupling a Nucleoside or Nucleoside Analog to a Polypeptide

In some approaches, the tagged polypeptide is obtained by coupling or attaching a nucleoside or nucleoside analog to a polypeptide. Any suitable method can be used to couple a nucleoside or nucleoside analog to a polypeptide. For example, the nucleoside or nucleoside analog may be attached to a crosslinker that contains a reactive moiety capable of forming a covalent bond to specific functional groups on the polypeptide. Crosslinkers and reactive moieties useful for coupling the nucleoside or nucleoside analog to a polypeptide are described, for example, in Hermanson (2013), “Bioconjugate Techniques”, 3^{rd} edition, Academic Press; and Mattson et al. (1993), “A practical approach to crosslinking, Mol. Biol. Rep., 17:167483. Exemplary crosslinkers that may be used include, but are not limited to, amine-reactive crosslinkers, carbonyl-reactive crosslinkers, carboxyl-reactive crosslinkers, sulfhydryl-reactive crosslinkers, and photo-reactive crosslinkers.

Amine-reactive crosslinkers contain a reactive moiety capable to form a bond with primary amines present on polypeptides, for example by either acylation or alkylation. Exemplary reactive moieties include, but are not limited to N-Hydroxysuccinimide (NHS) esters or sulfo-NHS esters, isothiocyanates, isocyanates, acyl azides, sulfonyl chlorides, aldehydes, glyoxals, epoxides, oxiranes, carbonates, aryl halides, imidoesters, anhydrides, carbodiimides, and fluorophenyl esters. In certain aspects, a nucleoside or nucleoside analog is coupled to a polypeptide through an amine-reactive crosslinker through an NHS ester.

Sulfhydryl-reactive crosslinkers contain a reactive moiety capable to form a bond with sulfhydryl present in the side chains of cysteine, usually by alkylation or disulfide exchange. Exemplary sulfhydryl-reactive moieties include, but are not limited to haloacetyls, maleimides, aziridines, acryloyls, arylating agents, vinylsulfones, pyridyl disulfides, TNB-thiols and disulfide reducing agents.

Carbonyl-reactive crosslinkers contain a reactive moiety capable to form a bond with carbonyl present on polypeptides (e.g. on glycoproteins). Exemplary reactive moieties include aldehyde- and ketone-reactive moieties like hydrazine, hydrazine derivatives, and alkoxyamines. Other carboxyl-reactive crosslinkers contain a reactive moiety capable to form a bond between carboxyls and primary amines. Exemplary reactive moieties include EDC, dicyclohexyl carbodiimide, and other carbodiimides.

Photo-reactive crosslinkers contain reactive moiety capable to form a bond with any functional group present on a polypeptide and that become reactive when exposed to ultraviolet or visible light. Exemplary photo-reactive moieties include aryl azide and diazirine.

### Antibodies and Other Binding Reagents

The technology put forth in this disclosure uses binding reagents that specifically bind to the nucleoside tag portion of the tagged polypeptide. In one aspect, the binding reagent binds to the nucleoside tag portion of the tagged polypeptide and does not bind to the polypeptide portion of the tagged polypeptide. In some approaches, the binding reagent does not bind to the polypeptide portion of the tagged polypeptide in the absence of the nucleoside tag portion. Accordingly, in some cases the binding reagent may bind or interact with parts of the polypeptide portion in the presence of the nucleoside tag portion. In some aspects, the binding reagent binds to the nucleoside tag of the tagged polypeptide and does not bind to an otherwise identical polypeptide without the nucleoside tag.

A binding reagent is a molecule or macromolecule that specifically recognizes and binds a nucleoside tag portion based on the structural feature of the nucleoside or nucleoside analog contained in the nucleoside tag portion. For example, a binding reagent may specifically bind to a nucleoside analog that comprises the nucleobase adenosine and an azidomethyl at the 3′ O-position of the sugar, but does not bind with high affinity to a nucleoside analog comprising the nucleobase adenosine but has a 3′ hydroxyl group instead of an azidomethyl and does not bind with high affinity to a nucleoside analog comprising the nucleobase cytosine, guanine, or thymine, each with or without an azidomethyl reversible blocking group. Accordingly, a binding reagent used in the method described herein may specifically bind to one nucleoside by recognizing its nucleobase (e.g., A) and not bind to a nucleoside comprising any other nucleobase (e.g., T, C and G).

Examples of binding reagents that may be used include antibodies (including antibody fragments, bispecific antibodies, Fab, Fab′, F(ab′)2, scFv, dsFv, ds-scFv, dimers, minibodies, nanobodies, diabodies, and multimers thereof, aptamers, knottins, affimers, or any other known reagent that binds a nucleoside or nucleoside analog with a suitable specificity and affinity. In some embodiments, the binding reagent is an antibody, such as a monoclonal antibody. In some embodiments, the binding reagent is an aptamer.

Antibodies that that specifically bind to the nucleosides or nucleoside analog can be obtained from a number of sources, such as LSBio, BML life, or Synaptic Systems. See also Feederle and Schepers (2017), “Antibodies specific for nucleic acid modifications”, RNA Biol., 14:1089-1098. Antibodies against nucleosides or nucleoside analogs for use as binding reagents may also be generated. Standard procedures for generating, purifying and modifying antibodies may be used and are described in e.g., Harlow and Lane (1988), “Antibodies, A Laboratory Manual”, CSH Press, Cold Spring Harbor; and Howard and Bethell (2000), “Basic Methods in Antibody Production and Characterization”, CRC Pr. Inc.). Phage display methods may also be used and are described, for example, in Ward et al. (1989), Nature, 341:544-546, 1989; Huse et al. (1989), Science 246:1275, 1989; and McCafferty et al. (1990), Nature 348:552-554; and in U.S. Pat. Nos. 5,403,484, 5,969,108 and 5,885,793. Useful antibodies may also be produced in a cell-free system. Non-limiting exemplary cell-free systems are described, e.g., in Sitaraman et al. (2009), Methods Mol. Biol., 498: 229-44; Spirin (2004), Trends Biotechnol., 22: 538-45, 2004; and Endo et al. (2003), Biotechnol. Adv., 21: 695-713.

Particular methods for raising and using antibody specific for nucleoside analogs are detailed in published international application WO 2018/129214 (“Drmanac et al. 2018”) and WO 2020/097607 (“Drmanac et al. 2020”).

In some embodiments, the binding reagent is an aptamer. Nucleic acid aptamers can be engineered through repeated rounds of in vitro selection or equivalently, SELEX (systematic evolution of ligands by exponential enrichment) to bind to various targets (such as nucleosides or nucleoside analogs). See, e.g., Jayasena, et al., Clinical Chemistry 45:1628-1650, 1999. Peptide aptamer selection can be made using different systems, including the yeast two-hybrid system. Peptide aptamers can also be selected from combinatorial peptide libraries constructed by phage display and other surface display technologies such as mRNA display, ribosome display, bacterial display and yeast display. These experimental procedures are also known as biopannings. See, e.g., Reverdatto et al., 2015, Curr. Top. Med. Chem. 15:1082-1101.

In some aspects, the binding reagent is an affimer. Affimers specific for a nucleoside or nucleoside analog can be selected by the use of phage display libraries that are screened to identify an affimer protein with high-specificity binding to the target nucleoside or nucleoside analog and high binding affinities. See, e.g., U.S. Pat. Nos. 8,481,491, 8,063,019, and WO 2009/136182, which are incorporated herein by reference. See also Crawford et al., Brief Funct. Genomic Proteomic, 2:72-79, 2003. In some aspects, the binding reagent is a knottin or inhibitor cystine knot (ICK), a protein structural motif containing three disulfide bridges. New binding epitopes can be introduced into natural knottins using protein engineering. One approach to production of knottins that are specific for a nucleoside or nucleoside analog is to create and screen knottin libraries using yeast surface display and fluorescence-activated cell sorting (see, e.g., Kintzing and Cochran, Curr. Opin. Chem. Biol. 34:143-150, 2016; Moore et al., Drug Discovery Today: Technologies 9(1): e3-ell, 2012; and Moore and Cochran, Meth. Enzymol. 503:223-51, 2012).

### Preparation and Use of Removable Antibodies

Removal of anti-nucleoside binding reagents is described in published international application WO 2018/129214 (“Drmanac et al. 2018”) and WO 2020/097607 (“Drmanac et al. 2020”), both incorporated herein by reference. See, e.g., Sections 4.6, 8.1.2-8.1.3, Examples 11-12 of Drmanac 2018 (for example and not for limitation) and 0207, 0213-0223, 0228-0231 of Drmanac et al. 2020. Drmanac 2018 and 2020 describe disassociation conditions for removing anti-nucleoside affinity reagents from nucleosides incorporated into primer extension products. However, these conditions will also disassociate affinity reagents from tagged polypeptides. Exemplary conditions for removal may be high temperature, such as temperature in the range from 50° C. to 75° C., and sometimes 55° C. to 75° C., e.g., 60° C. to 70° C. In some embodiments, the high pH is greater than 7, greater than 8, e.g., about 9. Exemplary dissociation conditions comprise a high pH environment (pH in the range from pH 8 to 10). In preferred embodiments the dissociation conditions comprise high temperature and high pH. Additionally, in some embodiments, removal of the antibody occurs in a reaction mixture that contains salt at a concentration that is less than 100 mM, such as less than 90 mM, or less than 80 mM. Under preferred disassociation conditions the antibody removal or dissociation generally can be carried out within less than 60 seconds, e.g., less than 40 seconds, or less than 30 seconds. In some embodiments, the dissociation conditions are those under which at least about 90%, sometimes at least about 95%, and sometimes at least about 99% of the bound labeled antibodies are dissociated from the tagged protein in less than 5 minutes, less than 60 seconds, e.g., less than 40 seconds, or less than 30 seconds, less than 20 s, or less than 10 s. In contrast, binding conditions suitable for antibody-antigen interaction. For example, in some embodiments, binding occurs at a temperature that in the ranges of 30 to 45° C. or 35-50° C. In some embodiments, binding occurs in an environment having a pH that ranges from 7 to 8.5, often 7 to 7.5. In some embodiments, binding is performed binding conditions include a temperature in the range from 30-45° C. and/or an environment having a pH that ranges from 7 to 7.5. It will be recognized that binding reagents (e.g., antibodies) that are removable (can be removed from the will be selected in part based on the binding characteristics. As described below, antibodies that can be removed are selected. Although monoclonal antibodies with a high equilibrium dissociation constant (KD), are preferred for some applications, lower affinity antibodies are preferred in detection procedures where the antibody is to be removed.

Multiplex immunochemistry and other uses of this technology may include a removal step in which a set of tag-specific antibodies used in a first staining reaction is removed from a biological sample, thereby permitting the sample to be subjected to a second round of staining. The antibodies in the first staining reaction are chosen such that they may be removed from the sample under mild conditions, thereby preserving the tissue.

To obtain suitable removable antibodies using hybridoma technology, the user injects mice or rabbits with the nucleoside or nucleoside analog (the “epitope”) on a carrier protein such as keyhole limpet hemocyanin (KLH), in the usual fashion, recovers lymphocytes, and makes hybridomas. Typically an initial screening of the hybridomas is done in the usual fashion to obtain hybridoma clones that are specific for the respective epitope. A second round of screening is done to obtain the removable antibodies by contacting each of the clones with the corresponding epitope. After binding the epitope, the clones are subject to treatment under the same or similar conditions to what will be used for removal: for example, a solution containing the nucleoside or nucleoside analog in a moderate salt solution.

Clones that are positive in the first screening but negative after exposure to the removal solution are selected as candidates having the desired epitope specificity with a low to moderate affinity, suitable for removal.

Removal of low-affinity antibodies from tagged proteins on cells or in tissue samples is performed under dissociation conditions wherein the bound, labelled antibodies are dissociated from the tagged protein. Exemplary dissociation conditions comprise a high temperature, such as temperature in the range from 50° C. to 75° C., and sometimes 55° C. to 75° C., e.g., 60° C. to 70° C. In some embodiments, the high pH is greater than 7, greater than 8, e.g., about 9. Exemplary dissociation conditions comprise a high pH environment (pH in the range from pH 8 to 10). The dissociation conditions may include a concentration of the same nucleoside or nucleoside analog to which the antibody is specific in free form, to help remove the antibody by binding site competition. The dissociation conditions can be set such that at least about 80%, 90%, or 95% of the bound antibodies are dissociated from the sample in less than 5 minutes.

Exemplary monoclonal antibodies that bind specifically to 3′-azidomethyl nucleosides are described in Drmanac 2020 (WO 2020/097607) including without limitation monoclonal antibodies specific for: 3′-azidomethyl-dA (N3A): mAbs 2C5, 3B12, 17H7, and 18B7); monoclonal antibodies specific for 3′-azidomethyl-dC (N3C): mAbs 1B8, 2B9, 4C8, 1A10, and 3B7; monoclonal antibodies specific for 3′-azidomethyl-dG (N3G): mAbs 3G6, 5F6, 4B8, 4G8, and 7C8; and monoclonal antibodies specific for 3′-azidomethyl-dT (N3T): mAbs 2D4, 2D10, 1F9, and 3B7. Exemplary polyclonal and monoclonal antibodies have been made that bind specifically to natural (no 3′ blocking group) nucleosides and nucleoside. This includes the analog 3′-nitrobenzyloxo or 3′-nitromethylbenzyloxo, described in Example 2, below. Binding specificity was confirmed in assays using the respective nucleoside or nucleoside analog conjugated to biotin or to a protein such as bovine serum albumin.

### Detectable Labels

In some approaches, the binding reagent is conjugated to a detectable label. The binding reagent can be labelled using any methods known in the art. Methods for linking of antibodies and other binding reagents to detectable labels, e.g., signal-generating proteins including enzymes and fluorescent/luminescent proteins are well known in the art (Wild, The Immunoassay Handbook, 4.sup.th ed.; Elsevier: Amsterdam, the Netherlands, 2013; Kobayashi and Oyama, Analyst 136:642-651, 2011). Any suitable label that generates a signal and is detectable may be used. In some embodiments, the detectable label is a fluorescent dye or fluorophore.

Methods for conjugating a fluorescent label to a binding reagent and a variety of fluorescent molecules, and their optical properties are described in, e.g., Haugland (2005), Handbook of Fluorescent Probes and Research Chemicals (Molecular Probes, Eugene). Exemplary fluorescent dyes include, without limitation, acridine dyes, cyanine dyes, fluorone dyes, oxazine dyes, phenanthridine dyes, and rhodamine dyes, e.g., fluorescein, FITC, Texas Red, ROX, cyanine 3 (Cy 3), cyanine 5 (Cy 5), Alexa Fluor dye (e.g., Alexa Fluor 647 or 488), ATTO dye (e.g., ATTO 532 or 655).

In some approaches, the detectable label is a chemiluminescent (e.g., bioluminescent) label. In some embodiments, the chemiluminescent label is an enzyme, that produces a detectable signal in the presence of substrate. Exemplary enzymes that may be used as labels include, but are not limited to luciferase, horseradish peroxidase, beta-galactosidase, and alkaline phosphatase. Enzyme based labels are described e.g., in Rashidian et al. (2013), Bioconjugate Chem. 24:1277-1294.

### Direct and Indirect Labelling Methods

In some approaches, the binding reagent is directly labelled, e.g., the binding reagent is conjugated to a detectable label, for example via a covalent bond. In some approaches, indirect labelling methods are used to detect binding of the binding reagent to the nucleoside portion of the tagged polypeptide. Accordingly, in some embodiments, the binding reagent is a primary binding reagent and directly binds the nucleoside portion of the tagged polypeptide. The primary binding reagent may be unlabelled, e.g. the primary binding reagent is not conjugated to a detectable label. Detection of the binding of the primary bind reagent to the nucleoside tag portion of the polypeptide occurs through a secondary binding reagent that binds the primary binding reagent. In certain embodiments, the secondary antibody contains an antigen binding region which specifically binds to the primary antibody, e.g., the constant region of the primary antibody. The secondary binding reagent may be conjugated to a detectable label. Thus, detecting the binding of the primary binding reagent to the nucleoside tag portion of the tagged polypeptide comprises (i) contacting the primary binding reagent with a secondary binding reagent, where the secondary binding reagent is conjugated to a detectable label, and where the secondary binding reagent binds to the primary binding reagent; and (ii) detecting the binding of the secondary binding reagent to the first binding reagent comprising detecting a signal from the detectable label conjugated to the secondary binding reagent. In some aspects, the primary binding reagent is a primary antibody. In some aspects, the secondary binding reagent is a secondary antibody.

### Detection Methods

Detecting binding of a binding reagent to the nucleoside tag portion of a tagged protein can be done by detecting a signal from a detectable label attached to the binding reagent. Detecting a signal from the detectable label may be performed by a variety of methods, and selection of a suitable detection method may be based on the nature of the label (e.g., fluorophore, chemiluminescent agent etc.). For example, the signal may be detected visually using, e.g., light microscopy, fluorescent microscopy, electron microscopy, spectrophotometry.

Complexes that Comprise Binding Reagents and Tagged Polypeptides

Aspects of this technology further relate to a complex of a binding reagent and a tagged polypeptide, where the tagged polypeptide includes a polypeptide portion and a nucleoside tag portion, and the binding reagent is bound to the nucleoside tag portion of the tagged polypeptide. In some embodiments, the binding reagent is not bound to the polypeptide portion of the tagged polypeptide. In some embodiments, the binding reagent that is bound to the nucleoside tag portion of the tagged polypeptide is an antibody or aptamer. In some embodiments, the binding reagent is a primary binding reagent (e.g., a primary antibody). In some embodiments, the binding reagent-tagged polypeptide complex is recognized and bound by a secondary binding reagent (e.g., a secondary antibody) that is conjugated to a detectable label. In some aspects, the secondary binding reagent binds the primary binding reagent.

In some approaches, the method described herein is used to detect one or more polypeptides in a sample. A sample can be any solid or fluid sample obtained, for example, from animals, plants, bacteria, yeast, protozoans, or amoebas. For example, a sample can be a biological sample obtained from any organ or tissue (including a biopsy or autopsy specimen, such as a tumor biopsy) or can include a cell (whether a primary cell or cultured cell) or medium conditioned by any cell, tissue or organ. In some embodiments, the biological sample is a tissue sample. A sample can also be a biological fluid such as urine, cerebrospinal fluid, blood, lymph fluids, tissue homogenate, interstitial fluid, cell extracts, mucus, saliva, sputum, stool, physiological secretions or other similar fluids. In some embodiments, the sample comprises cell culture derived cells.

### Kits

In some aspects, the technology provides kits that are configured for practicing the method described herein. In some embodiments the kit comprises (a) a tagged polypeptide comprising a polypeptide portion and a nucleoside tag portion, and (b) a binding reagent that specifically binds the nucleoside tag portion of the tagged polypeptide. The kit may comprise 2, 3, 5, or a plurality of such combinations of tagged polypeptide with the corresponding binding reagent. In some embodiments, the binding reagent is an antibody. In some embodiments, the binding reagent is conjugated to a detectable label. In some embodiments, the detectable label is a fluorescent or a chemiluminescent label. In some embodiments, the binding reagent is a primary binding reagent and the kit further comprises a secondary binding reagent that specifically binds to the primary binding reagent. In some embodiments, the secondary binding reagent is conjugated to a detectable label. In some embodiments, the detectable label is a fluorescent or a chemiluminescent label.

In some aspects the technology provides a kit comprising a binding reagent-tagged polypeptide complex. In some embodiments, the binding reagent is a primary binding reagent. In some embodiments, the kit further comprises a secondary binding reagent that specifically binds the primary binding reagent of the binding reagent-tagged polypeptide complex.

### Definitions

In some parts of the description above, the binding reagent is referred to as an antibody that specific for the target nucleoside or nucleoside analog. In this sense, the term “antibody” is used as a non-limiting illustration of a binding reagent. In each of the illustrations, he term “antibody” is interchangeable with the term “binding reagent” as a more general category unless otherwise stated or required.

The terms “polypeptide,” “peptide,” and “protein” are used interchangeably herein to refer to a polymer of amino acids. As used herein, the terms encompass amino acid chains of any length, including full-length proteins, wherein the amino acid residues are linked by covalent peptide bonds.

As used herein a “nucleoside” generally refers to a naturally occurring nucleoside comprising a naturally occurring nucleobase (or nitrogenous base) linked to a five-carbon sugar. A “naturally occurring nucleobase,” as used herein, means adenine (A), cytosine (C), guanine (G), thymine (T), or uracil (U). Five-carbon sugars include ribose and 2′-deoxyribose. A “naturally occurring nucleoside,” as used herein, refers to deoxyadenosine, deoxycytidine, deoxyguanosine, deoxythymidine, adenosine, cytidine, guanosine, and thymidine.

As used herein, a “nucleoside analog” refers to a compound or molecule whose core structure is the same, or closely resembles that of a nucleoside, but generally differs from a nucleoside by a modification of the nucleobase, modification of sugar, and/or the addition of a phosphate moiety to the sugar. Modifications may include substitutions or the addition of a chemical moiety.

As used herein, a “nucleotide” refers to a nucleoside comprising a phosphate moiety of one or more phosphates (e.g., a monophosphate, a diphosphate, or a triphosphate consisting of one, two, or three linked phosphates, respectively). The phosphate moiety is usually attached to the 5-carbon of the sugar.

The term “binding reagent” as used herein refers to a molecule that binds to a nucleoside or nucleoside analog with desired affinity or specificity. In certain embodiments, the binding reagent is a first binding reagent that directly binds a nucleoside or nucleoside analog. In certain embodiments, the binding reagent is a secondary binding reagent which binds the first binding reagent.

The term “antibody” as used herein refers to an immunoglobulin molecule or composition (e.g., monoclonal and polyclonal antibodies), as well as genetically engineered forms such as chimeric, humanized and human antibodies, heteroconjugate antibodies (e.g., bispecific antibodies), and antibody fragments. The antibody may be from recombinant sources and/or produced in animals, including without limitation transgenic animals. The term “antibody” as used herein includes “antibody fragments,” including without limitation Fab, Fab′, F(ab′)₂, scFv, dsFv, ds-scFv, dimers, minibodies, nanobodies diabodies, and multimers thereof and bispecific antibody fragments. The antibodies can be in any useful isotype, including IgM and IgG, such as IgG1, IgG2, IgG3 and IgG4. In some embodiments, the antibody is a primary antibody that specifically binds a nucleoside or nucleoside analog. In some embodiments, the antibody is a secondary antibody that specifically binds the primary antibody.

As used herein, the term “aptamer” refers to refers to a small molecule that can bind specifically to another molecule (e.g., a nucleoside or nucleoside analog). Aptamers are typically either polynucleotide- or peptide-based molecules. A polynucleotidal aptamer is a DNA or RNA molecule, usually comprising a (usually short) strand of oligonucleotides. Peptide aptamers which comprise one (or more) short variable peptide domains, attached at both ends to a protein scaffold. Polynucleotidal aptamers are usually engineered through repeated rounds of in vitro selection or equivalently, SELEX (systematic evolution of ligands by exponential enrichment) to bind to a target.

As used herein, the term “detectable label” refers to any atom or molecule that can be used to provide a detectable and/or quantifiable signal. Suitable labels include fluorophores, molecules that emit chemiluminescence, enzymes, enzyme substrates, chromophores, radioisotopes, mass labels, electron dense particles, magnetic particles, spin labels, electrochemically active molecules, and cofactors. The detectable label may be conjugated directly or indirectly to another molecule (e.g., a binding reagent) to facilitate detection of that molecule.

The term “coupling” or any grammatical variation thereof (e.g., couple, coupled, etc.) as used herein refer to any chemical association between two molecules and include both covalent and non-covalent associations. In one embodiment, the term coupling means a covalent association between a polypeptide and a nucleoside or nucleoside analog.

As used herein, the term “reactive moiety” means a part or portion of a molecule that may react to form one or more bonds with another molecule. For example, the reactive group might be an amine-reactive group, such as an NHS ester, where the ester reacts with amines present on polypeptides.

As used herein, the term a “sample” can be any solid or fluid sample obtained from, excreted by or secreted by any living organism, including without limitation, animals, plants, bacteria, yeast, protozoans, and amoebas. For example, a sample can be a biological sample obtained from any organ or tissue (including a biopsy or autopsy specimen, such as a tumor biopsy) or can include a cell (whether a primary cell or cultured cell) or medium conditioned by any cell, tissue or organ. A sample can also be a biological fluid obtained from, for example, blood, plasma, serum, urine, bile, ascites, saliva, cerebrospinal fluid, or any other bodily secretion.

As used herein, the terms “alkyl,” “alkenyl,” and “alkynyl” include straight- and branched-chain monovalent substituents. Examples include methyl, ethyl, isobutyl, 3-butynyl, and the like. “Heteroalkyl,” “heteroalkenyl,” and “heteroalkynyl” are defined similarly as alkyl, alkenyl, and alkynyl, but can contain O, S, or N heteroatoms or combinations thereof within the backbone.

As used herein, the term “substituted” includes the addition of an alkoxy, aryloxy, amino, alkyl, alkenyl, alkynyl, aryl, heteroalkyl, heteroalkenyl, heteroalkynyl, heteroaryl, cycloalkyl, or heterocycloalkyl group to a position attached to the main chain of the alkoxy, aryloxy, amino, alkyl, alkenyl, alkynyl, aryl, heteroalkyl, heteroalkenyl, heteroalkynyl, heteroaryl, cycloalkyl, or heterocycloalkyl, e.g., the replacement of a hydrogen by one of these molecules. Examples of substitution groups include, but are not limited to, hydroxy, halogen (e.g., F, Br, CI, or I), and carboxyl groups. Conversely, as used herein, the term “unsubstituted” indicates the alkyl, alkenyl, alkynyl, heteroalkyl, heteroalkenyl, or heteroalkynyl has a full complement of hydrogens, i.e., commensurate with its saturation level, with no substitutions, e.g., linear butane (—(CH₂)₃—CH₃).

### Practice of the Invention

All of the products and methods claimed in this disclosure may be used for any suitable purpose without restriction, unless otherwise indicated or required. While the invention has been described with reference to the specific examples and illustrations, changes can be made and equivalents can be substituted to adapt the technology to a particular context or intended use as a matter of routine development and optimization and within the purview of one of ordinary skill in the art, thereby achieving benefits of the invention without departing from the scope of what is claimed and their equivalents.

### EXAMPLES

### Example 1: Synthesis of Nucleosides or Nucleoside Analogs Comprising a N-Hydroxy-Succinimide (NHS) Reactive Moiety

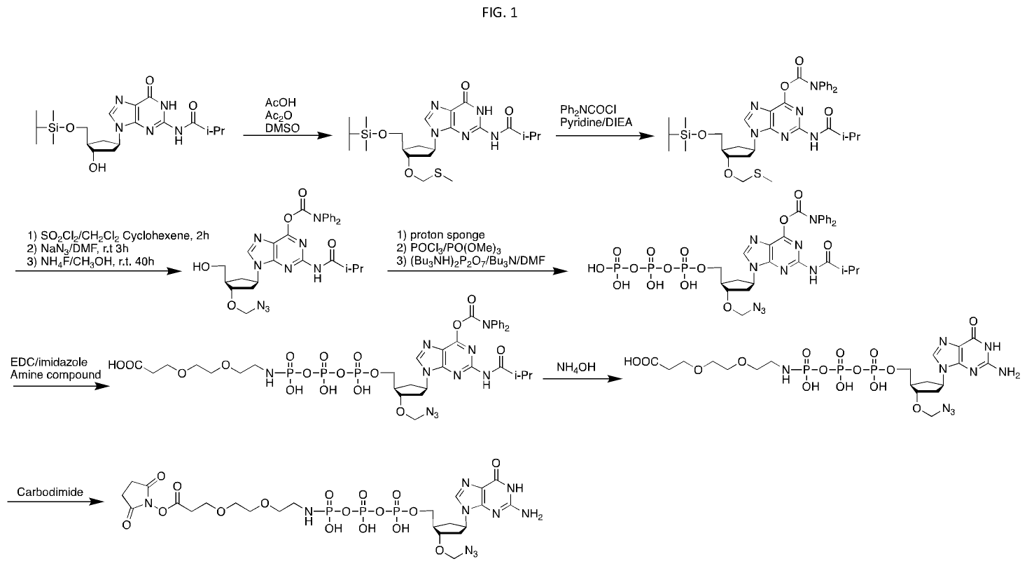

The synthesis of the amino-reactive N-hydroxysuccinimide (NHS) ester of a nucleoside analog comprising an 3′-O azidomethyl moiety is shown in FIG. 1. This equips the analog with a leaving group, so that the nucleoside analog can be conjugated to a chosen protein.

Using this chemistry, proteins can be tagged by reaction of available free amines on the protein with NHS ester activated nucleosides. NHS ester modified nucleosides are diluted in anhydrous DMSO, and reacted with the protein at concentrations that provide sufficient tagging without adversely affecting protein function. Relatively low concentrations of protein are adjusted to pH 8.0 in bicarbonate buffer, and then reacted with the NHS ester nucleosides. Incubation typically continues for 60 min at room temperature or lower temperatures, before separation of the tagged protein from unused tagging reagent and byproducts.

### Example 2: Production and Characterization of Antibody

Specific antibodies have been generated against various nucleosides and nucleoside analogs for use according to this disclosure.

Rabbit monoclonal antibodies were raised against KLH-conjugated 3′-azidomethyl-dA (N3A), 3′-azidomethyl-dC (N3C), 3′-azidomethyl-dG (N3G), or 3′-azidomethyl-dT (N3T) (Yurogen Biosystems, Worcester, MA). Briefly, rabbits were immunized with four different nucleoside analogs conjugated to keyhole limpet hemocyanin (KLH). Bleed analysis by ELISA was performed using each NLRT. On day 63 post-immunization, rabbits were sacrificed and peripheral blood mononuclear cells (PBMC) or splenocytes were isolated. Rabbits were selected for cell sorting and culturing antibody-secreting B-cells. The co-culture supernatants were screened using the nucleoside analogs used for immunization. Selected clones were cultured, antibody was harvested from the culture supernatant, and purified according to standard protocols.

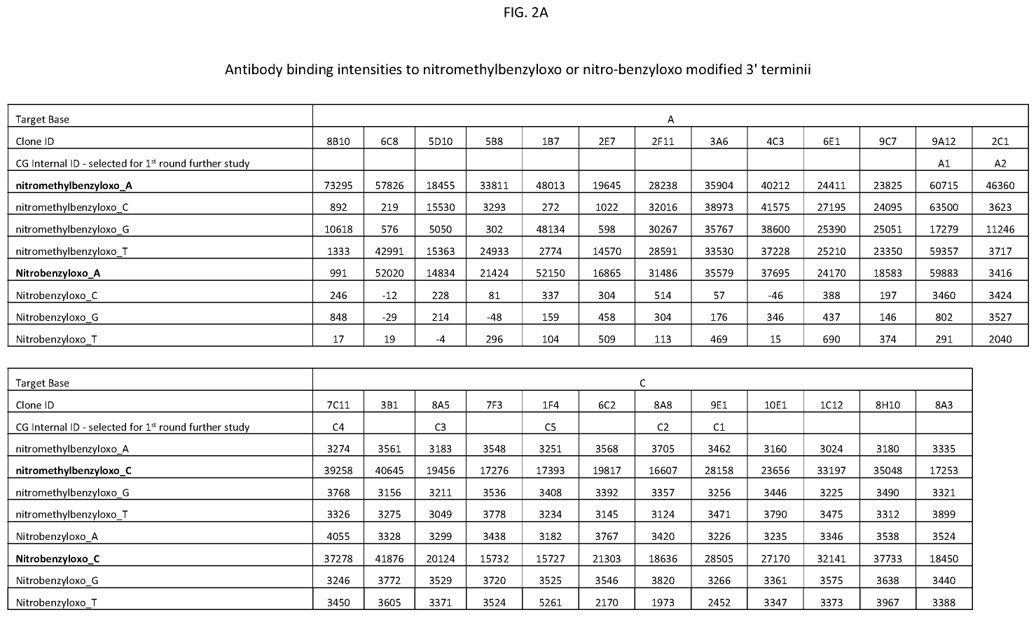

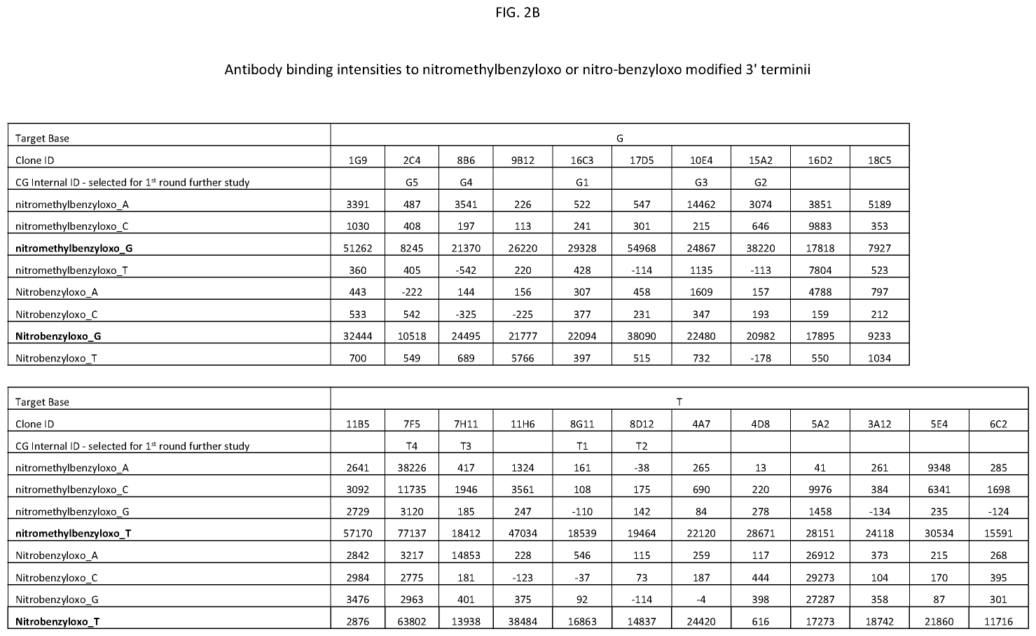

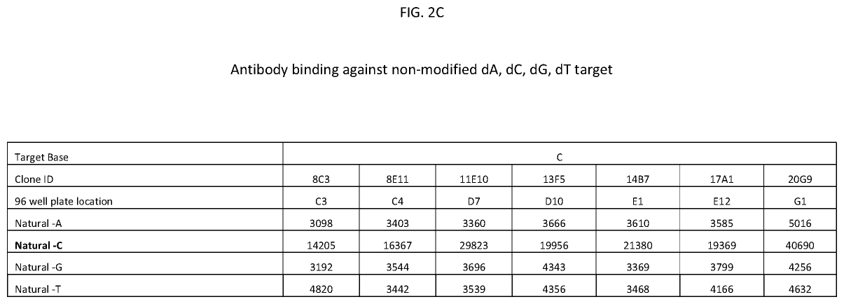

FIGS. 2A, 2B, and 2C provide data characterizing the binding specificity of such antibodies. Polyclonal antibodies were obtained from isolated B-cell supernatants. Antibody preparations are identified in the table by laboratory designation (“clone ID”). Antibody binding intensity is shown for 3′-nitrobenzyloxo or 3′-nitromethylbenzyloxo modified nucleotides incorporated onto a primer. Non-modified terminal nucleotides were used to detect natural C binding antibodies. In this context, “binding intensity” refers to the level of primary antibody binding as reflected in the level of fluorescently labeled anti-rabbit IgG secondary antibody measured using fluorescence mode of a plate reader. Four different primers were used, which incorporated four different terminal modified nucleotides (A, T, C, and G) which were then tested against the respective antibody. Primary antibodies were detected with a fluorescently labelled anti Rabbit IgG secondary antibody. Specific binding to the target and off-target bases were determined with a non-competing binding event.

The assay was conducted as follows: A 3′ biotinylated oligonucleotide was attached to commercially available streptavidin coated 96-well microtiter plates. A primer was then hybridized to the biotinylated template strand attached to the well of the plate. A 3′ modified reversible terminator nucleotide was then incorporated to the first position after the 3′ end of the primer as directed by the template strand position directly opposite. Four primers were selected such that the first base of incorporation resulted in all 4 nucleobases (A, C, G, T) being incorporated in different wells of the plate. After incorporation of the nucleotide, culture supernatant of immunized rabbit B-cell clones was incubated in the wells of the plate for 5 min at 37° C. to allow binding of positive antibodies to the terminal base of the primer. After a wash with buffered salt solution, a commercially available fluorescently labeled secondary goat anti-rabbit IgG antibody (AAT biosciences) was used to detect the presence or absence of binding of the B-cell supernatant derived rabbit IgG.

### INCORPORATION BY REFERENCE

For all purposes, each and every publication and patent document cited in this disclosure is hereby incorporated herein by reference in its entirety for all purposes to the same extent as if each such publication or document was specifically and individually indicated to be incorporated herein by reference.

1. A method of determining location of a protein in a biological sample, comprising:
attaching a tag to the protein to form a tagged protein, wherein the tag is a nucleoside or nucleoside analog;
contacting the biological sample with the tagged protein; then
identifying and determining location of the protein in the sample by contacting the sample with a binding reagent that binds specifically to the tag.

2. The method of claim 1, wherein the tag is a nucleoside or analog thereof.

3-4. (canceled)

5. The method of
claim 2, wherein said tag is a nucleoside comprising at least one modification compared with a nucleoside of the following formula:

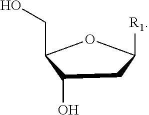

6. The method of claim 3, wherein the nucleoside analog is a naturally occurring nucleoside modified at the 3′ —OH of the nucleoside.

7. The method of claim 1, wherein the binding reagent comprises a fluorescent group.

8. The method of claim 7, wherein location of the tagged protein in the sample is determined by obtaining an image of fluorescence emitted from the florescent group.

9. The method of claim 1, which is a method of flow cytometry or cell sorting, wherein the biological sample is a sample of cells.

10. The method of claim 1, which is a method of immunohistochemistry, wherein the biological sample is a tissue sample.

11. The method claim 1, which is a method of enzyme linked immuno-assays (ELISA), or a method of producing a western blot.

12. The method of claim 1, which is a multiplex method.

13. The method of claim 1, which is a method for tracking a plurality of proteins in vivo.

14. A method of tracking a plurality of proteins in vivo, the method comprising:
obtaining a plurality of proteins, each tagged with a different nucleoside or nucleoside analog;
administering the tagged proteins to a subject either as a mixture, simultaneously, or sequentially; then
obtaining a biological sample from the subject; and
determining the presence, quantity, and/or location of each of the tagged proteins in the biological sample by contacting the biological sample with binding reagents specific for each of the tags on the tagged proteins.

15. The method of claim 14, wherein the biological sample is a tissue section, and each of the specific binding reagents is used to determine location of the respective tagged proteins in the tissue section.

16. The method of claim 15, wherein the determining is a multiplex method in which at least some of the tagged proteins have been tagged with two or more different nucleoside or nucleoside analogs, wherein a particular combination of the nucleoside or nucleoside analogs uniquely identifies each of the tagged proteins.

17. The method of claim 15, wherein the determining is a multiplex method that includes several iterations, wherein the method comprises:
contacting the tissue section with a set of tag-specific binding reagents that collectively bind some but not all of the different nucleoside tags attached to proteins in the tissue section;
locating tagged proteins in the tissue section that bear tags for which the binding reagents in the set are specific;
removing the binding reagents from the tissue sample; and
performing one or more additional iterations of the contacting, locating, and removing using a different set of binding reagents that contains at least one binding reagent that is specific for a tag that is different from the tags to which the binding reagents were specific in preceding iteration(s).

18. The method of claim 17, wherein the binding reagents are removed using a solution that contains a non-physiological salt concentration and/or pH, and one or more nucleosides or nucleoside analogs that are not attached to proteins.

19. The method of claim 17, wherein the binding reagents used to contact the tissue section in each set are fluorescent, and the determining in each set includes obtaining an image of emitted fluorescence from binding reagents bound to the tissue section.

20. The method of claim 19, wherein four different proteins are located during each iteration using four fluorescent binding reagents that are specific for each of the tags attached to the four different proteins, wherein fluorescent emission from each of the binding reagents is optically distinguishable from fluorescent emission from the other binding reagents.

21. The method of claim 19, further comprising computationally merging images obtained from each of the iterations.

22. The method of claim 14, wherein the biological sample is a liquid sample, and the presence and/or quantity of tagged protein in the sample is determined using an array of binding reagents on a solid surface, wherein separate position on the array each comprise a binding reagent specific for a different tag.

23. The method of claim 1, wherein the binding reagent(s) are antibodies, nanobodies, antibody fragments, or affimers.

24-26. (canceled)

27. The method of claim 1, wherein identity, quantity, and/or location of tagged protein in the sample is confirmed by contacting the sample with a binding reagent that binds specifically to its respective tag on the tagged protein, both before and after treating the tag chemically or enzymatically to remove the tag from the protein or modifying the tag to a form not recognized by the binding reagent.
